(19) United States
(12) Patent Application Publication
(10) Pub. No.: US 2020/0179160 A1
(43) Pub. Date: Jun. 11, 2020

(54) RADIOLUCENT MEDICAL TABLE HEATING PAD

(71) Applicant: Cray Villaflor NOAH, Spring, TX (US)

(72) Inventor: Cray Villaflor NOAH, Spring, TX (US)

(21) Appl. No.: 16/608,766

(22) PCT Filed: Apr. 25, 2018
(86) PCT No.: PCT/US2018/029465

§ 371 Date: Oct. 25, 2019

Related U.S. Application Data
(60) Provisional application No. 62/489,581, filed on Apr. 25, 2017.

(51) Int. Cl. A61F 7/00; H05B 3/14; H05B 3/34

(52) U.S. Cl. CPC A61F 7/007; A61G 13/10; H05B 3/34; H05B 3/145

(57) ABSTRACT

A device for warming a medical patient includes a first layer and a radiolucent resistive heating element. The first layer is sized to be positioned under a medical patient. The radiolucent resistive heating element is proximate to the first layer and is configured to generate a thermal energy to warm the first layer to at least 37° C.

### CROSS-REFERENCE TO RELATED APPLICATIONS

This application claims the benefit of and priority to U.S. Provisional Patent Application No. 62/489,581, filed on Apr. 25, 2018, which is hereby incorporated by reference in its entirety.

### BACKGROUND

It has long been known the human body functions best at a temperature of 37° C. This is especially the case in the hospital, where intraoperative hypothermia or more simply, getting cold during surgery—severely jeopardizes patient health. Even mild hypothermia, defined as a core body temperature below 36° C., prevents blood from clotting, weakens the immune system, and increases recovery time—burdening both patients and hospitals. Unfortunately, combatting intraoperative hypothermia is no easy task for anesthesiologists. Patients get cold quickly during surgery for three main reasons: they are naked or wearing only a thin gown; open wounds and incisions release large amounts of heat to the environment; and operating rooms (ORs) are kept cold for sterility purposes.

Hundreds of conversations with medical professionals and firsthand exposure in hospital ORs show a clear consensus: anesthesiologists lack an adequate solution to prevent intraoperative hypothermia. The current standard forced-air warming blankets may create difficulties for the many surgical procedures involving large body area and/or air flow may contaminate the sterile zone. The less frequently used conductive heating pads and blankets contain metal, which affects x-ray and/or other imaging, which is an increasingly prevalent technology used in ORs. Despite a solution for intraoperative hypothermia being such a long felt need, intraoperative hypothermia is reported in over half of all surgical cases.

### BRIEF SUMMARY

In some embodiments, a device for warming a medical patient includes a first layer and a radiolucent resistive heating element. The first layer is sized to be positioned under a medical patient. The radiolucent resistive heating element is positioned below the first layer and is configured to generate a thermal energy to warm the first layer.

In other embodiments, a medical table for warming a medical patient includes a table and a warming pad. The table includes an upper surface sized to support the medical patient on the upper surface. The warming pad is connected to the table and forms at least a portion of the upper surface of the table. The warming pad includes a first layer and a radiolucent resistive heating element. The first layer is sized to be positioned under a medical patient. The radiolucent resistive heating element is positioned below the first layer and is configured to generate a thermal energy to warm the first layer to at least 37° C.

In yet other embodiments, a method of warming a medical patient includes generating a thermal energy with a resistive heating element, conducting the thermal energy through an uncompressed conformable layer at a first conduction rate, compressing at least a portion of the uncompressed conformable layer to form a compressed portion of the conformable layer, and conducting the thermal energy through the compressed portion at a second conduction rate that is different from the first conduction rate.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the disclosure may be realized and obtained by means of the instruments and combinations particularly pointed out in the appended claims. Features of the present disclosure will become more fully apparent from the following description and appended claims or may be learned by the practice of the disclosure as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1 is an exploded view of a warming pad, according to at least one embodiment of the present disclosure;

FIG. 2 is a schematic cross-sectional view of thermal energy conducted through the warming pad of FIG. 1, according to at least one embodiment of the present disclosure;

FIG. 3 is a graph illustrating example input voltages to heat a warming pad versus time, according to at least one embodiment of the present disclosure;

FIG. 4 is a perspective view of a medical table with a warming upper surface, according to at least one embodiment of the present disclosure;

FIG. 5 is a top view of a warming pad illustrating different warming areas, according to at least one embodiment of the present disclosure;

FIG. 6 is a top view of a medical table illustrating different warming areas, according to at least one embodiment of the present disclosure; and

FIG. 7 is a flowchart illustrating a method of warming a patient, according to at least one embodiment of the present disclosure.

### DETAILED DESCRIPTION

This disclosure generally relates to devices, systems, and methods for warming a medical patient. More particularly, the present disclosure relates to maintaining the comfort and wellbeing of a patient on a medical table during imaging and other medical procedures. In some embodiments, a warming pad may include a radiolucent resistive heating element. For example, the heating element may be a sheet or a plurality of threads or wires of a conductive heating material with a resistivity sufficient to dissipate input current as thermal energy. The heating material, while conductive and capable of producing thermal energy, may be substantially radiolucent. In other words, the resistive heating element may be transparent in a source energy range used during radiology or other radio-imaging of a patient, human or animal, positioned on the warming pad.

In some embodiments, the radiolucent resistive heating element may have a radiopacity of less than 100 Hounsfield units (where 0 Hounsfield units is the radiopacity of water, and conventional copper wires have a radiopacity of 14,000 Hounsfield units or more). A radiopacity of less than 100 Hounsfield units may allow the imaging of soft tissue without interference from the radiolucent resistive heating element. In other embodiments, the radiolucent resistive heating element may have a radiopacity of less than 0 Hounsfield units. A radiopacity of less than 0 Hounsfield units may allow the imaging of bodily fluids without interference from the radiolucent resistive heating element. In some embodiments, the radiolucent resistive heating element may be made of a radiolucent heating material (i.e., the material that generates the thermal energy may be radiolucent). For example, the heating element material may have a radiopacity of less than 100 Hounsfield units. In other examples, the heating element material may have a radiopacity of less than 0 Hounsfield units.

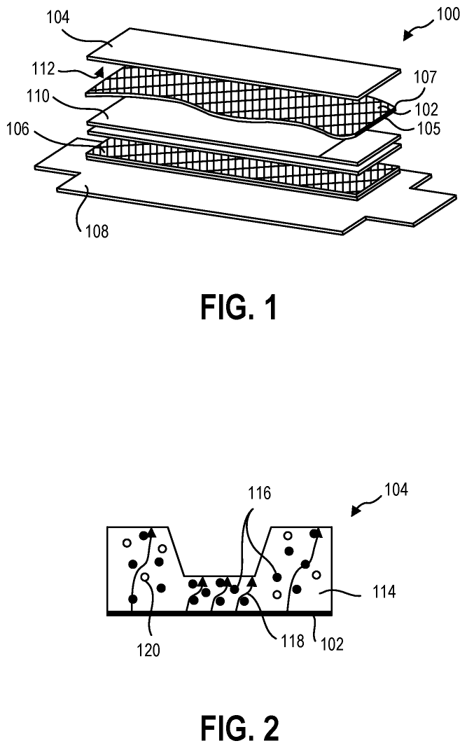

FIG. 1 is an exploded view of an embodiment of a warming pad 100 according to the present disclosure. The warming pad 100 includes a radiolucent resistive heating element 102 in the warming pad 100. The radiolucent resistive heating element 102 generates a thermal energy when an electrical current and/or electrical voltage is provided to the radiolucent resistive heating element 102. At least a portion of the thermal energy is thermally conducted through a first layer to a patient or other object placed on the warming pad 100. In some embodiments, the first layer may be a conformable layer 104.

In some embodiments, the radiolucent resistive heating element 102 may be free of metal to allow the radiolucent resistive heating element 102 to be transparent during radiology or other imaging of a patient on or under the warming pad 100. For example, the radiolucent resistive heating element 102 may include carbon fiber as the heating material. In some examples, the radiolucent resistive heating element 102 may be a carbon fiber fabric. Carbon fiber fabric is entirely radiolucent and may reach temperatures up to 110° C. without material fatigue or failure. In some examples, carbon fiber fabric may have a resistivity of about 0.1 Ohms per meter. Testing indicates a resistivity of carbon fiber fabric is unaffected by changes in temperature within the range of 20-105° C. In other examples, other carbon-based materials may be used. In other embodiments, the warming pad 100 may include a metal electrode 105 or other contact, such as a silver contact along an edge 107 of the radiolucent resistive heating element 102, to provide electrical communication to the radiolucent resistive heating element 102.

In some embodiments, at least portions of the conformable layer 104 may be thermally conductive to conduct the thermal energy from the radiolucent resistive heating element 102 toward a patient. For example, the conformable layer 104 may be an insulating material that is doped with thermally conductive particles. In at least one example, the conformable layer 104 may include a polymer, such as a memory foam, that is doped with graphite particles. The conformable layer 104 may include a closed-cell foam with air pockets contained and sealed within the foam.

In some embodiments, the conformable layer 104 may have a thermal conductivity of at least 1.0 cal/gram° C. In other embodiments, the conformable layer 104 may have a thermal conductivity of at least 1.5 cal/gram° C. In yet other embodiments, the conformable layer 104 may have a thermal conductivity of at least 2.0 cal/gram° C.

The warming pad 100 may further include a comfort layer 106 that includes a deformable material to provide the patient with additional padding and comfort during use. In some embodiments, the comfort layer 106 may include or be doped with thermally conductive particles. In at least one example, the comfort layer 106 may include a foam that is doped with graphite particles. The comfort layer 106 may include a closed-cell foam with air pockets contained and sealed within the foam. The thermally conductive material of the comfort layer 106 may have a thermal conductivity that is less than a thermal conductivity of the conformable layer 104. In at least one embodiment, the comfort layer 106 may conduct thermal energy away from the radiolucent resistive heating element 102 to limit or prevent overheating of the radiolucent resistive heating element 102.

In some embodiments, the warming pad 100 may include a medical upholstery layer 108 on a bottom and/or a top surface of the warming pad 100. The medical upholstery layer 108 may be a treated polyvinyl chloride (PVC) polyester blend. The medical upholstery layer 108 may be nonporous and can be sterilized without material fatigue. The medical upholstery layer 108 may encase all other layers of the warming pad 100 and may be sewn shut with internal seams.

In some embodiments, the warming pad 100 may include a thermally insulating layer 110 positioned under the radiolucent resistive heating element 102 to resist heat flow downward (i.e., toward the comfort layer 106) and help to direct thermal energy produced by the radiolucent resistive heating element 102 upwards towards the patient.

In some examples, the thermally insulating layer 110 may include polyvinyl chloride (PVC). For example, in testing of warming pad designs according to the present disclosure, PVC has a specific heat of 0.20-0.29 cal/gram° C., compared to 0.43 cal/gram° C. for conventional memory foam, and 1.5 cal/gram° C. for the graphite-doped foam used in some embodiments of the conformable layer 104 and/or comfort layer 106.

In some embodiments, to increase thermal conductivity and thermal contact between the conformable layer 104 and the radiolucent resistive heating element 102, a conductive gel 112 may be positioned between the conformable layer 104 and radiolucent resistive heating element 102.

The conformable layer 104 may have a thermal conductivity that changes with compression of the conformable layer 104, such as a patient lying on the warming pad 100. For example, FIG. 2 illustrates the conformable layer 104 of FIG. 1 in an uncompressed state and a compressed state. Upon compression from the uncompressed state toward the compressed state, the foam 114 may reduce in a vertical dimension, moving the thermally conductive particles 116 closer together and reducing the size of the air pockets 120. This may, in turn, increase the thermal conductivity from the radiolucent resistive heating element 102 through the thermally conductive particles 116 and reduce the insulating effects of the air pockets 120.

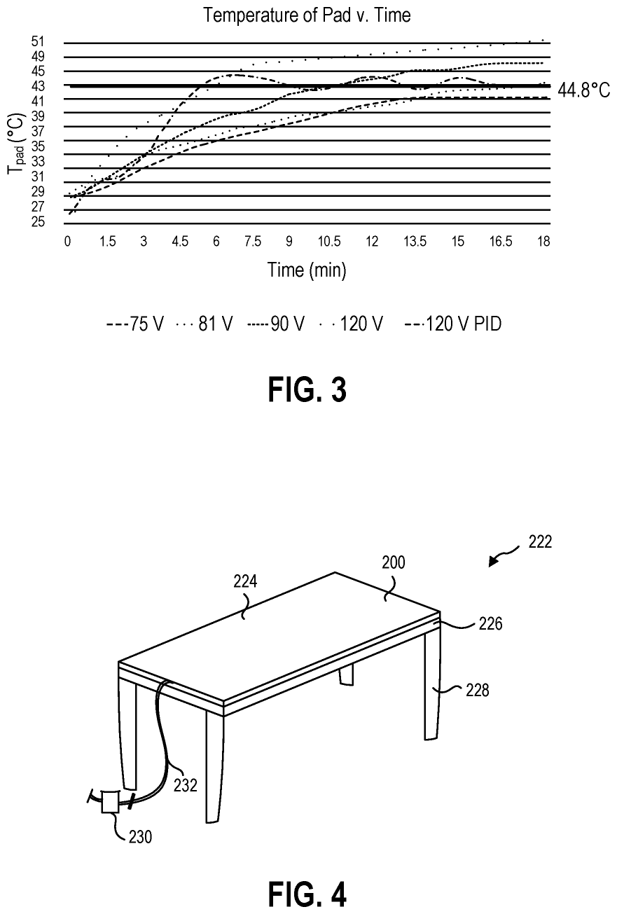

In some embodiments, the radiolucent resistive heating element may receive an input current and/or voltage. FIG. 3 is a graph illustrating the temperatures of a warming pad over time with different input voltages and a constant 1 ampere (A) of input current.

In some embodiments, the warming pad may provide thermal energy to warm a patient to at least 37° Celsius (° C.). For example, the radiolucent resistive heating element may attain a temperature of at least 37° C. In other examples, the radiolucent resistive heating element may attain a temperature greater than 37° C. to provide sufficient thermal energy through the other layers of the warming pad and maintain a contact surface of the patient at a temperature at least 37° C. In some embodiments, a heating element temperature of between 37° C. and 50° C. may maintain a contact surface with a patient at a temperature at least 37° C.

A warming pad, according to at least one embodiment of the present disclosure, may attain 44.8° C. with an input voltage between 81 Volts (V) and 120 V. An input voltage of 81 V, according to at least one embodiment of the present disclosure, may attain 44.8° C. in approximately 16.5 minutes. An input voltage of 120 V, according to at least one embodiment of the present disclosure, may attain 44.8° C. in approximately 5 minutes. It may desirable to achieve 44.8° C., and hence a patient temperature of 37° C., in a shorter period of time, however, the warming pad temperature may continue to rise beyond 44.8° C. with an input voltage of 120 V. In particular, warming pad temperatures above 46° C. may injure a patient, and, in some embodiments, it may be critical to maintain the warming pad surface below 46° C.

In some embodiments, a warming pad may include a controller or other feedback device to adjust the input voltage to maintain a predetermined heating element temperature. For example, a PID (Present/Integral/Derivative) controller may employ one or more feedback loops to respond in real time to deviations in the target temperature. In some embodiments, a controller may maintain a quasi-equilibrium in a range around the target temperature. For example, the controller may allow fluctuations around the target temperature of no more than 1.5° C. In other examples, the controller may allow fluctuations around the target temperature of no more than 1.0° C. In yet other examples, the controller may allow fluctuations around the target temperature of no more than 0.5° C. For example, FIG. 3 illustrates a curve of a PID controller with up to 120 V of input voltage that allows the warming pad to attain the target temperature (44.8° C.) in approximately 5 minutes, and subsequently reduces the input voltage to keep the heating element temperature at or near the target temperature.

FIG. 4 is a perspective view of a medical table 222 with a warming pad 200 integrated into the medical table 222, according to at least one embodiment of the present disclosure. For example, the medical table 222 may have an upper surface 224 upon which a patient may sit or lie. In some embodiments, at least a portion of the upper surface 224 may be formed by a warming pad 200. For example, a medical table 222 may have a support surface 226 that provides a lower surface of the warming pad 200 such that at least one layer of the warming pad (the radiolucent resistive heating element, the conformable layer, the medical upholstery layer, the comfort layer) may be affixed to the support surface 226. In some embodiments, the medical table 222 may have legs 228 to support the support surface 226, such as an examination table. In other embodiments, the medical table 222 may have wheels affixed to the support surface 226 to allow the medical table 222 to be moved easily, such as an operating bed.

FIG. 4 further illustrates an embodiment of a controller 230 that may control the input voltage and/or input current through an electrical conduit 232 to the radiolucent resistive heating element. In some embodiments, the controller 230 may be a PID controller, such as described in relation to FIG. 3. In other embodiments, the controller 230 may be another input current and/or voltage control mechanism, such as a switch (or switch array) or a variable resistor. For example, the controller 230 may be a dial that a user may set to a desired input voltage. In other examples, the controller 230 may be an array of switches that allows selection of an array of resistors to select predetermined input voltages.

In some embodiments, the conduit 232 may be a conventional electrical wire, such as aluminum or copper wire. In other embodiments, the conduit 232 may be a non-metallic conduit such as a carbon-based conductor. For example, the conduit 232 may include graphite, graphene, carbon fiber, carbon fiber fabric, or other conductive nonmetals. In some embodiments, the electrical conduit 232 and the medical table 222 (including the warming pad 200) may be free of any metals, allowing the medical table 222 and/or warming pad 200 to be used in a magnetic resonance imaging environment without interacting with the magnetic field. In other embodiments, the electrical conduit 232 may be selectively removable from the warming pad 200 and/or the medical table 222 such that the warming pad 200 and/or the medical table 222 may be used with in a magnetic resonance imaging environment and the thermal energy retained in the warming pad 200 and/or medical table 222 may keep the patient warm for some time during magnetic resonance imaging.

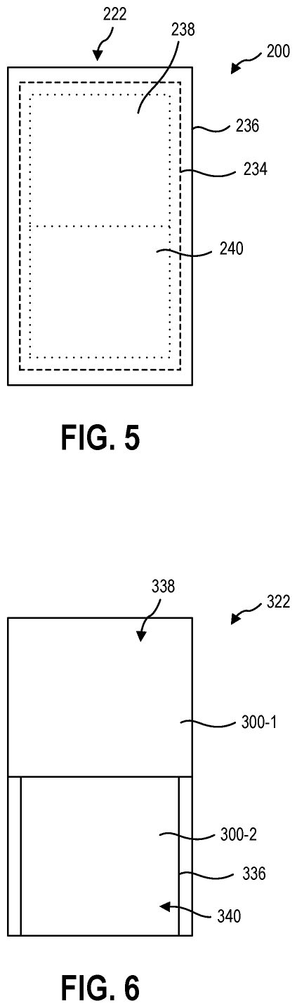

While FIG. 4 illustrates a medical table 222 with a warming pad 200 forming the upper surface 224, in some embodiments, the medical table 222 and/or warming pad 200 may be configured to provide thermal energy to an area less than the entire upper surface 224. For example, FIG. 5 is a top view of the medical table 222 of FIG. 4. In some embodiments, the warming pad 200 may be configured to be radiolucent and/or free of metal in an area less than the entire surface of the warming pad 200.

For example, a warming pad 200 may be configured to be radiolucent and/or free of metal in a patient portion 234 of the warming pad 200 and/or medical table 222. In other words, the warming pad 200 may have an electrical contact, such as silver, that is located around a border 236 of the warming pad 200. The border 236 may contain metal that allows for an even electrical voltage and/or current to be delivered to the radiolucent resistive heating element, while the metal in the border 236 does not interfere with imaging of a patient positioned on the patient portion 234 of the warming pad 200. In some examples, the border 236 may be less than 1 centimeter (cm) in width from the edge of the warming pad 200. In other examples, the border 236 may be less than 3 cm in width from the edge of the warming pad 200. In yet other examples, the border 236 may be less than 5 cm in width from the edge of the warming pad 200.

In other embodiments, a warming pad 200 may be radiolucent and/or free of metal in an upper body portion 238 and/or a lower body portion 240 of the warming pad 200. For example, a medical table 222 may be configured to be specific to imaging of a patient's chest cavity, and the warming pad 200 may be radiolucent and/or free of metal in the upper body portion 238.

In other embodiments, a medical table may have a plurality of warming pads. FIG. 6 is a top view of another embodiment of a medical table 322 with a first warming pad 300-1 in an upper body portion 338 and a second warming pad 300-2 in a lower body portion 340 of the medical table 322. In some embodiments, the first warming pad 300-1 and second warming pad 300-2 may be controlled (i.e., temperatures adjusted) independently. In other embodiments, the first warming pad 300-1 and second warming pad 300-2 may have different thicknesses of padding. In yet other embodiments, the first warming pad 300-1 may be radiolucent and free of metals, while the second warming pad 300-2 may be radiolucent in part, with metal in a border 336. In yet other embodiments, a medical table and/or warming pad may have one or more radiolucent resistive heating elements configured in other manners.

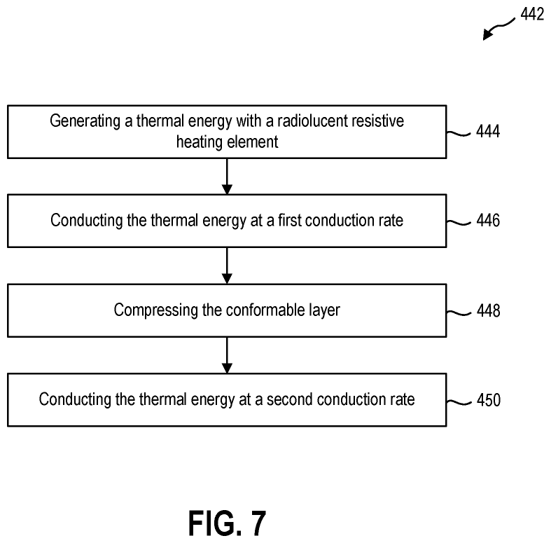

FIG. 7 is a flowchart illustrating an embodiment of a method of warming a patient, according to the present disclosure. In some embodiments, the method 442 may include generating a thermal energy with a radiolucent resistive heating element at 444 and conducting the thermal energy with an uncompressed conformable layer at a first conduction rate at 446. The method 442 further includes compressing the conformable layer at 448 to produce a compressed portion of the conformable layer. The compressed portion may have a second thermal conduction rate that is greater than the first thermal conduction rate. The method 442 may further include conducting the thermal energy at the second conduction rate at 450 with the compressed portion. In some embodiments, the thermal conduction rate may change because of an increase in thermal conductivity of the conformable layer. For example, compression may move thermally conductive particle closer together, increasing thermal conductivity. In other embodiments, the thermal conduction rate may change because of a decrease in thermal insulation of the conformable layer. For example, compression may move compress air pockets or other insulating portions of the conformable layer, reducing the insulating characteristics of the conformable layer. In such embodiments, the method 442 may provide a reactive warming pad that selectively provides thermal energy at a greater rate to the portions of the warming pad compressed by a patient's presence.

The method may include transportation of a patient while in contact with the warming pad. For example, the patient may be moved from the operating room to an imaging room, imaging may be performed, and the patient may be moved back to the operating room without removing the user from the warming pad.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

It should be understood that any directions or reference frames in the preceding description are merely relative directions or movements. For example, any references to “above” and “below” or “top” and “bottom” or “left” and “right” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A device for warming a medical patient, the device comprising:
a first layer, the first layer being sized to be positioned proximate the medical patient; and
a radiolucent resistive heating element positioned in or below the first layer, the radiolucent resistive heating element configured to generate a thermal energy to warm the first layer.

2. The device of claim 1, the radiolucent resistive heating element including carbon fiber.

3. The device of any preceding claim, the radiolucent resistive heating element being free of any metallic components.

4. The device of any preceding claim, further comprising an electrical conduit in communication with the radiolucent heating element.

5. The device of claim 4, the electrical conduit being free of any metallic components.

6. The device of claim 4, the electrical conduit being selectively removable from the radiolucent heating element.

7. The device of any preceding claim, the first layer further including a thermally conductive padding to conduct the thermal energy from the radiolucent resistive heating element toward an upper surface of the first layer.

8. The device of claim 7, the thermally conductive padding having a thermal conductivity that increases when the thermally conductive padding is compressed.

9. The device of claim 7, the thermally conductive padding including a graphite-doped polymer.

10. The device of any preceding claim, further comprising a feedback controller in electrical communication with the radiolucent heating element to control a temperature of the radiolucent heating element.

11. The device of any preceding claim, further comprising a comfort layer positioned on an opposite side of the radiolucent resistive heating element from the first layer.

12. The device of any preceding claim, the first layer being a conformable layer.

13. A medical table for warming a medical patient, the table comprising:
a table having an upper surface sized to support the medical patient on the upper surface;
a warming pad connected to the table and forming at least a portion of the upper surface of the table, the warming pad including:
a first layer, the first layer being sized to be positioned under at least a portion of the medical patient; and
a radiolucent resistive heating element positioned in below the first layer, the radiolucent resistive heating element configured to generate a thermal energy to warm the first layer to at least 37 degrees Celsius.

14. The table of claim 13, the radiolucent resistive heating element including carbon fiber.

15. The table of claim 13 or 14, the first layer having a thermal conductivity that increases when the thermally conductive padding is compressed.

16. The table of any of claims 13 through 15, the warming pad being sized to allow it to be draped over or around a portion of the medical patient.

17. The table of any of claims 13 through 16, the warming pad being free of metal.

18. A method of warming a medical patient, the method comprising:
generating a thermal energy with a resistive heating element;
conducting the thermal energy through an uncompressed conformable layer at a first conduction rate;
compressing at least a portion of the uncompressed conformable layer to form a compressed portion of the conformable layer; and
conducting the thermal energy through the compressed portion at a second conduction rate that is greater than the first conduction rate.

19. The method of claim 18, the resistive heating element being radiolucent.

20. The method of claim 18 or 19, generating the thermal energy including providing no more than 1 ampere of current to the radiolucent resistive heating element.
